(19) United States
(12) Patent Application Publication
(10) Pub. No.: US 2021/0260580 A1
(43) Pub. Date: Aug. 26, 2021

(54) Method for monitoring COVID-19

(71) Applicant: Dawn Bennett, Burlington, NJ (US)

(72) Inventor: Dawn Bennett, Burlington, NJ (US)

(21) Appl. No.: 17/240,082

(22) Filed: Apr. 26, 2021

Related U.S. Application Data
(63) Continuation-in-part of application No. 15/976,937, filed on May 11, 2018, now abandoned.
(60) Provisional application No. 62/508,149, filed on May 18, 2017.

(51) Int. Cl. B01L 3/00; G01N 27/447; G01N 33/536; G01N 1/40

(52) U.S. Cl. CPC B01L 3/502715; G01N 27/447; G01N 33/536; G01N 2001/4038; B01L 2200/027; B01L 2300/0636; B01L 2400/0421; G01N 1/40

(57) ABSTRACT

A method of sampling and testing for SARS-COV-2 virus in nasal and nasopharyngeal fluid using a plurality of microfluidic channels with a plurality of integrated electrodes in the microfluidic channels to detect the virus. In one example embodiment, a plurality of antibodies are fixed on a surface of at least one electrode by positive dielectrophoresis that increases the sensitivity of detection. Viral antigens bind to the antibodies separating from the fluid thereby signally that the virus is present as evidenced by the detection of the antigens. Sampling by microfluidic channels is more comfortable to a patient because microfluidic channels are soft, flexible and narrow compared to swabs. Another example embodiment of a method using microfluidic channels for collecting tears or saliva to determine blood glucose levels using a smartphone that has been modified to incorporate external filters quantitate glucose levels is also described.

### CROSS-REFERENCE TO RELATED APPLICATIONS

This application is a continuation-in-part of the nonprovisional utility application, Ser. No. 15/976,937, filed in the United States Patent Office on May 11, 2018, claiming the priority to the provisional patent application, Ser. No. 62/508,149, filed in the United States Patent Office on May 18, 2017 and claims the priority thereof and is expressly incorporated herein by reference in its entirety.

### TECHNICAL FIELD

The present disclosure relates generally to a diagnostic method. More particularly, the present disclosure relates to a method for monitoring the presence of the SARS CoV-2 virus in bodily fluids, especially nasal fluids as well as monitoring other conditions such as blood glucose levels.

### BACKGROUND

The recent pandemic that began in 2020 caused by the spread of the SARS-COV-2 virus, formally known as severe acute respiratory syndrome coronavirus 2, causing the coronavirus disease known as COVID-19, has resulted in a growing need for a pain-free method of extracting bodily fluids from the nasal wall and oropharynx and a rapid and accurate means of detecting the virus in the bodily fluids. Millions of people died worldwide while the disease rapidly spread throughout the world.

The disease was easily spread by infected people who were asymptomatic or had not yet developed typical symptoms because the incubation period was as long as 10 days. For asymptomatic carriers of the virus and people in the early stage of infection prior to the onset of symptoms, the only method of detection was through sampling and testing of naso- and nasopharyngeal fluid.

There were many disadvantages to these early test methods. Results took days, placing people waiting for results in quarantine. The tests were in short supply and some versions were unreliable. Additionally, the sampling method of inserting a hard swab into the nasopharynx or the middle turbinate was uncomfortable.

Travel halted, businesses were shuttered, unemployment rose, and economies were greatly injured because of the necessity for people to avoid public places, travel and in person interactions with people outside their immediate household to prevent the further spread of the disease. Without a quick method to determine who was infected or a carrier, the world was unable to avoid the risk of the virus spreading without these harsh measures.

There are diagnostic needs that remain to be addressed. Diabetes is a disorder of the metabolism in which the body does not produce or properly use insulin. As a result, blood glucose levels must be monitored in order to control sugar spikes and initiate insulin injection to prevent hyperglycemia.

The incidence of diabetes has increased in the United States recently and it is projected to continue to increase in the future. While research continues both on prevention and cure, the patient with diabetes has no alternative but to learn how to control his or her blood glucose levels through diet, exercise and medication. Maintaining safe blood glucose is imperative for minimizing the debilitating side effects of diabetes.

It is imperative for Type 1 (insulin dependent) patients, especially to know what their blood glucose levels are to avoid going into diabetic shock (hypoglycemia), which can be life-threatening or experience the plurality of symptoms caused by too high sugar (extreme hyperglycemia). Type 2 or non-insulin dependent patients must also monitor their glucose levels to make sure that diet, exercise and oral medication is maintaining a glucose level that does not exceed the safe upper limit. Key to maintaining a safe blood glucose level is monitoring that level during the day and adjusting exercise, medication and diet.

Most people find managing diabetes expensive, uncomfortable and often overwhelming. Current methods of measuring blood glucose levels include pricking the finger or wearing a continuous glucose monitoring (“CGM”) system with an insulin pump. Finger pricking is unpleasant and can be painful. If used over a long period of time, finger pricking has a risk of infection and can cause damage to the finger tissue.

Continuous glucose monitoring systems are expensive, costing thousands of dollars. CGM systems use subcutaneous sensors to determine glucose levels in interstitial fluid and require a procedure to implant the sensors. However, CGM systems must be supplemented with finger pricking, at least 3 to 4 times daily, and finger pricking can be painful. These sensors require frequent calibration and cannot be used for more than a few days, as the sensors are prone to befouling. Any change in insulin dose must be preceded with figure pricking and confirmed by meter readings. In addition, existing glucose monitors fail to provide sufficient warning about the direction and history regarding potential hypoglycemia and extreme hyperglycemia in order to make proper insulin injections.

Because the majority of patients with diabetes (>50%) find managing the disease exhausting, there is a tremendous opportunity for less painful, less complicated noninvasive technologies for monitoring blood glucose.

Other proposed methods include the ocular glucose monitor, which uses laser light with chemical binding of ligands and analytes and the Tear TOUCH glucose biosensor, which uses enzymatic detection of glucose are both more technologically complex and, therefore, costlier.

Most of the existing noninvasive technologies that involve optical sensing are sensitive to temperature, pressure, the environment, interference from biological compounds and water content in blood, and they have poor signal to noise ratios. Others require extracting tears using a glass capillary which has great potential for damaging the eye.

While these methods may be suitable for many patients, they would not be as suitable for the purposes of the present disclosure as disclosed hereafter.

In the present disclosure, where a document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

An aspect of an example embodiment in the present disclosure is to provide a sampling method for SARS-COV-2 virus and similar pathogens that is more comfortable for a patient. Accordingly, an aspect of an example embodiment in the present disclosure provides a microfluidic device that is narrower than a swab and constructed from soft, flexible materials that are more comfortable for a patient than a swab.

Another aspect of an example embodiment in the present disclosure is to provide a rapid method of detecting SARS-COV-2 virus and similar pathogens. Accordingly, another aspect of an example embodiment in the present disclosure is to provide an electrode within a microfluidic system that uses positive dielectrophoresis that increases the sensitivity of detection and further provides quick results.

An aspect of an example embodiment in the present disclosure is to provide a non-invasive method of measuring blood glucose levels. Accordingly, an aspect of an example embodiment in the present disclosure provides a method of measuring blood glucose levels that does not require a finger prick or a subcutaneous sensor.

Another aspect of an example embodiment in the present disclosure is to provide a method of measuring blood glucose levels that uses accessible bodily fluids to determine blood glucose levels. Accordingly, the present disclosure provides a method of measuring blood glucose levels that uses tears or saliva to determine blood glucose levels.

A further aspect of an example embodiment in the present disclosure is to provide a method of measuring blood glucose levels that uses fluorescence technology. According, the present disclosure provides a method of measuring blood glucose levels using a fluorophore complex (combination of a fluorophore and glucose binding protein or other lectin) that is added to tear and saliva fluid to produce images that are quantitatively measure by fluorescence technology.

Yet another aspect of an example embodiment in the present disclosure is to provide a method of measuring blood glucose levels that electronically measures and stores a result in a modified smartphone or similar handheld personal computer. According, the present disclosure provides a method of measuring blood glucose levels using a smartphone or similar handheld personal computer that has been modified to incorporate external filters for the excitation of and transmission of emission fluorescence to measure glucose levels in tear fluid and saliva.

Accordingly, the present disclosure describes a method of sampling and testing for SARS-COV-2 virus in nasal and nasopharyngeal fluid using a plurality of microfluidic channels with a plurality of integrated electrodes in the microfluidic channels to detect the virus. In one example embodiment, a plurality of antibodies are fixed on a surface of at least one electrode by positive dielectrophoresis that increases the sensitivity of detection. Viral antigens bind to the antibodies separating from the fluid thereby signally that the virus is present as evidenced by the detection of the antigens. Sampling by microfluidic channels is more comfortable to a patient because microfluidic channels are soft, flexible and narrow compared to swabs.

Accordingly, the present disclosure describes a non-invasive method of measuring blood glucose levels that does not require a finger prick or a subcutaneous catheter. The method collects easily accessible bodily fluids such as tears or saliva to determine blood glucose levels. A fluorophore complex (combination of a fluorophore and glucose binding protein or lectin) is added to tear or saliva fluid to produce images that are quantitatively measured by fluorescence technology. A smartphone or similar handheld personal computer that has been modified to incorporate external filters for the excitation by light at a specific wavelength and emission of light at another wavelength measures the fluorophore complex to quantitate glucose levels in tear fluid and saliva. The measurement is made on a test strip that collects tears or saliva in one embodiment. In a further embodiment, the test strip is placed in a modified glucose meter that measures the fluorescence intensity and yields a digital reading of the glucose level.

In another embodiment, a biocompatible fluorophore complex is placed directly in the patient's eye and the patient holds the modified handheld personal computer in front of the eye and captures an image that is measured through the external filters. In other embodiments, fluorophore complexes that bind to amyloid-beta plaque, and to a plurality of other marker chemicals that predict Alzheimer's, chronic kidney and cardiovascular disease are combined with tears or saliva and measured using the modified handheld personal computer. In another embodiment, the test strip contains a fluidic channel.

The present disclosure addresses at least one of the foregoing disadvantages discussed hereinabove. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### BRIEF DESCRIPTION OF THE DRAWINGS

The drawings are briefly described as follows.

FIG. 1A is a top plan view of an example embodiment of a microfluidic device constructed from cellulosic material.

FIG. 1B is a top plan view of another example embodiment of a microfluidic device constructed from cellulosic material.

FIG. 1C is a top plan view of a further example embodiment of a microfluidic device constructed from cellulosic material.

FIG. 1D is a plan view of an example embodiment of a smartphone back equipped with a pair of fluorescence filters.

FIG. 1E is the empirical formula of Alizarin Red S.

FIG. 1F is the empirical formula of phenylboronic acid.

FIG. 2 is a block diagram of a method of measuring glucose levels using tear fluid.

FIG. 3 is a graph of fluorescence intensity versus glucose concentration.

FIG. 4 is a graph of tear glucose versus blood glucose.

FIG. 5 is a graph of fluorescence intensity while varying the concentration of phenylboronic acid and Alizarin Red S.

FIG. 6 is a perspective view of the example embodiment of a microfluidic device shown in FIG. 1A with a plurality of integrated electrodes.

FIG. 6A is a plan view of an example embodiment of a cross-section of the microfluidic device shown in FIG. 6.

FIG. 6B is a plan view of another example embodiment of a cross-section of the microfluidic device shown in FIG. 6.

FIG. 7 is a perspective view of the example embodiment of the microfluidic device shown in FIG. 6 coupled to a suction device.

FIG. 7A is a plan view of an example embodiment of a cross-section of the microfluidic device shown in FIG. 7.

FIG. 8 is a perspective view of the example embodiment of a microfluidic device shown in FIG. 1B with a plurality of channels.

FIG. 8A is a plan view of an example embodiment of a cross-section of the microfluidic device shown in FIG. 8.

FIG. 8B is a plan view of another example embodiment of a cross-section of the microfluidic device shown in FIG. 8.

FIG. 9 is a perspective view of the example embodiment of the microfluidic device shown in FIG. 8 coupled to a suction device.

FIG. 10 is a perspective view of the example embodiment of a microfluidic device shown in FIG. 10 with a plurality of channels.

FIG. 10A is a perspective view of an example embodiment of a cross-section of the microfluidic device shown in FIG. 10.

FIG. 11 is a perspective view of the example embodiment of the microfluidic device shown in FIG. 10 coupled to a suction device.

FIG. 12 is a plan view of an example embodiment of a triangular electrode paired with a curved electrode.

FIG. 12A is a plan view of an example embodiment of a cross-section of the triangular electrode shown in FIG. 12.

FIG. 12B is a plan view of an embodiment of a cross-section of the curved electrode shown in FIG. 12.

FIG. 12C is a perspective view of the example embodiment of the triangular electrode shown in FIG. 12.

FIG. 12D is a top plan view of the example embodiment of the curved electrode shown in FIG. 12.

FIG. 13 is a perspective view of the example embodiment of the triangular electrode paired with the curved electrode shown in FIG. 12 coupled to a suction device.

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete and fully conveys the scope of the present disclosure to those skilled in the art.

### DETAILED DESCRIPTION OF THE PREFERRED EMBODIMENTS

Herein disclosed is a system, a non-invasive method and a kit for measuring blood glucose levels in patients.

Glucose does not fluoresce under normal circumstances. In order for the system to measure the blood glucose level, bodily fluid is combined with a fluorophore complex (fluorophore and glucose-binding probe such as a lectin). The fluorophore complex combines with glucose in the sample, causing the glucose-fluorophore complex to absorb light at a known wavelength and emit light at a known wavelength.

In one embodiment, the sample is saliva. Collecting saliva is noninvasive, painless and easily collected. Saliva glucose levels are higher in type 2 patients than in type 1 patients. Saliva would be preferably tested in vitro using a suitable sampling device.

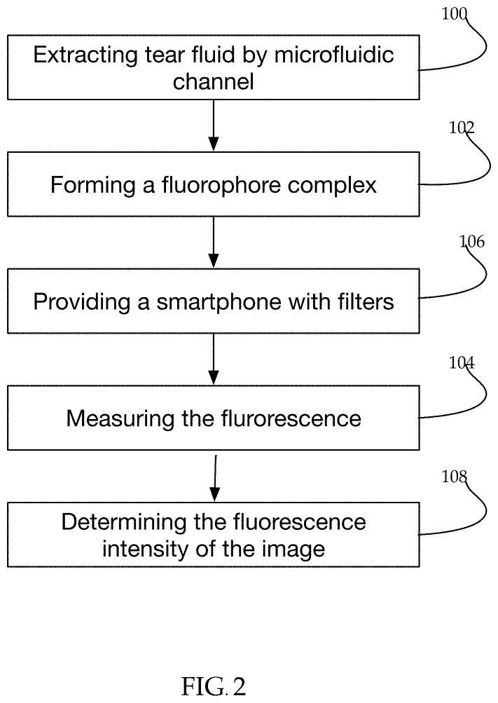

In a further embodiment, the sample is tear fluid. FIG. 2 shows the correlation between tear glucose levels and blood glucose levels.

Collecting tear fluid is highly advantageous. Tear fluid is highly accessible by non-invasive methods and can be continuously replenished by the lacrimal glands and other glands around the eyes so that the total volume of aqueous tear film layer is about 7 μl and the volume in the cul-de-sac about 4 μl. The average rate of tear fluid production ranges between 0.5 and 2.2 μl/min with an average of 1.2 μl/min. Tear fluid is maintained at a relatively stable volume of 4 μl. Analyzing glucose levels in tear fluid requires the ability to quantitatively measure very small sample volumes in a short time period by the methods described herein. Tears are more accessible than other bodily fluids, continuously obtainable, and less susceptible to dilution than urine. Moreover, tears are easier to access in public than urine. Determining glucose levels in tears and saliva both require high sensitivity and selectivity.

Providing a plurality of bodily fluid measurement methods allows a patient to select the method best suited for their needs. Tear fluid glucose analysis may be more suitable for type 1 patients as a preferred embodiment, where saliva analysis may be preferable for patients with dry or sensitive eyes.

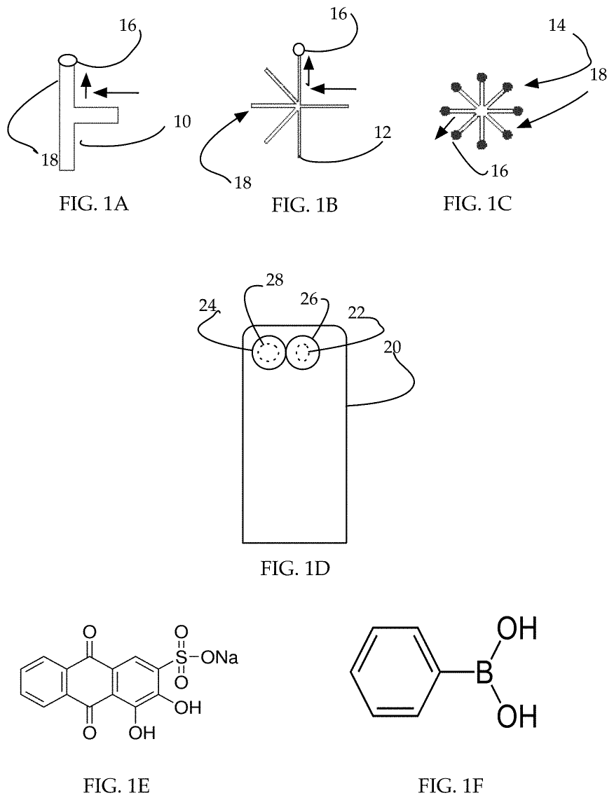

In the preferred example embodiment, the fluorophore glucose binding complex is Alizarin Red S shown in FIG. 1E and phenylboronic acid (PBA) shown FIG. 1F in phosphate buffer saline (PBS), forming a biocompatible fluorophore. A kit of this disclosure contains Alizarin Red S and phenylboronic acid (PBA) in phosphate buffer saline (PBS) as a preferred embodiment of the kit. In another embodiment, a lectin such as Concanavalin A (ConA) is employed.

In yet another example embodiment, colloidal gold nano-particles (AuNPs) with aminophenylboronic acid and fluorophores are an alternate method of developing the biosensor. It has been shown that the fluorescence of Rhodamine B isothiocyanate (RBITC) with AuNP's increases proportionally with the concentration of glucose in tear fluid at low glucose concentrations. The analysis can be performed using a small volume of fluid which is suitable for tear analysis because only a small volume of sample can be drawn at a time.

Other combinations of fluorophores and glucose binding complexes are possible within the inventive concept such as those listed below in Table 1.

TABLE 1

| | | |
|---|---|---|
| Texas Red→ Con A | Amplex → Red + GBP | Fluorescein |
| Alexa Fluor 488→ Con A | 2 NBDG + GBP | |
| Alexa Fluor 594 → Con A | Lissamine Green+ | |

In another embodiment of the method, a paper-based microfluidics sampling device shown in various embodiments FIGS. 1A-1C that collects the tear fluid and conveys the tear fluid into to a channel containing the fluorophore binding complex is placed in the eye lid pouch, and then measuring the fluorescence with the modified-handheld personal computer 20 shown in FIG. 1D.

The analysis of glucose levels in tear fluid is performed on microfluidic, paper-based devices shown in FIGS. 1A-1C. There are many advantages of using microfluidic systems. They require lower limits for detection, improve the precision of experiments, allow for running multiple assays for deletion, decrease sample and reagent consumption, and reduce the overall cost of applications.

The use of paper as a medium in the preferred example embodiment to construct microfluidic diagnostic systems also has important benefits. Paper is thin and lightweight; can be easily chemically modified with functional groups that bind to proteins, DNA and small molecules, and is light color and a good medium for calorimetric tests. Because of its chemical properties, cellulosic paper is extremely versatile and well-suited for application to microfluidics.

The microfluidic channels are designed with hydrophobic and hydrophilic barriers. The cellulosic paper is hydrophilic and is the region absorbing the material. The cellulosic paper is advantageously easily cut by laser to form microchannel configurations.

In other example embodiments, microfluidic devices constructed from softer biocompatible paper-based, synthetic hairs, cottons, and fabrics that can be placed in the lower eyelid pouch (lower fornix) and will not injure the patient's eye are within the inventive concept.

FIG. 1A shows an example embodiment of the device 10 with at least one microfluidic channel and three spokes 18. The arrows show the flow of the liquid. An analytical region 16 is where the fluorophore complex is mixed with tears. The microfluidic channels range from 300-550 μm. The length of each spoke 18 is approximately 1500 μm.

FIG. 1B shows a further example embodiment of the device 12 having a plurality of spokes. Similarly, the microfluidic channels range from 300-550 μm. and the length of each spoke 18 is approximately 1500 μm. The arrows show the flow of the liquid and the analytical region 16 indicates where the fluorophore complex is formed.

FIG. 1C shows another example embodiment of the device 14 having eight equidistant spokes 18 and the analytical region 16.

An embodiment of an in vitro method for determining blood glucose levels is illustrated in FIG. 2 and comprises sampling a bodily fluid, such as tear fluid 100, forming a fluorophore complex by mixing the bodily fluid with a fluorophore glucose-binding complex 102, and measuring the fluorophore-glucose level by fluorometric techniques 104. By providing using a handheld personal computer (smartphone) modified with appropriate light filters 106, the fluorescence can be measured. The software application translates fluorescence intensity of an image 108 into glucose levels is shown in FIG. 2.

The step of treating the device with a glucose-specific binding complex, forming a fluorophore complex 102 further comprises adding a combination of Alizarin Red S and phenylboronic acid to the device.

The step of measuring the fluorescence of the fluorophore complex is proceeded by the step of providing a handheld personal computer 106 equipped with a camera lens, a light source and a pair of filters, a first filter configured with the light source to provide light at the excitation energy of the fluorophore complex and a second filter configured to provide light at the emission wavelength of the fluorophore complex to the lens.

The step of measuring the fluorescence of the fluorophore complex 104 further comprises providing light through a first filter at the excitation energy of the fluorophore complex and measuring the light emitting from the device at the emission wavelength of the fluorophore complex.

The step of measuring the fluorescence of the fluorophore complex 104 further comprises capturing an image of the fluorescence by the camera of the handheld personal computer.

The step of measuring the fluorescence of the fluorophore complex 104 is followed by the step of determining the image fluorescence intensity to determine the concentration of the fluorophore complex 108.

The step of extracting the tear fluid from the patient's eye 100 uses a device having a paper-based microfluidic channel.

In one embodiment of the method a test strip, such as a Schirmer's test strip is placed in the lower eyelid pouch for a short time, extracting tear fluid onto the Schirmer's test strip, removing the strip from the eyelid pouch, adding fluorophore glucose binding complex to the strip and then measuring the fluorescence with the handheld personal computer modified with appropriate light filters and controlled by a software app that translates emission images on the strip into glucose levels.

In a further embodiment, the test strip is placed in a modified glucose meter that measures the fluorescence intensity and yields a digital reading of the glucose level.

In yet another embodiment, a biocompatible fluorophore complex (fluorophore and glucose binding protein or lectin) is placed directly in the tear fluid in the eye without collecting the tear fluid. The patient uses the handheld personal computer modified with appropriate light filters and controlled by a software app to capture an image of the eye by him- or herself that translates the emission images in the eye into glucose levels.

In yet a further embodiment of the method, a paper-based microfluidics sampling device shown in FIGS. 1A-1C collects saliva and conveys the saliva into at least one microfluidic channel containing the fluorophore binding complex, and then measuring the fluorescence with the handheld personal computer modified with appropriate light filters and controlled by a software app that translates emission images in the channel into glucose levels.

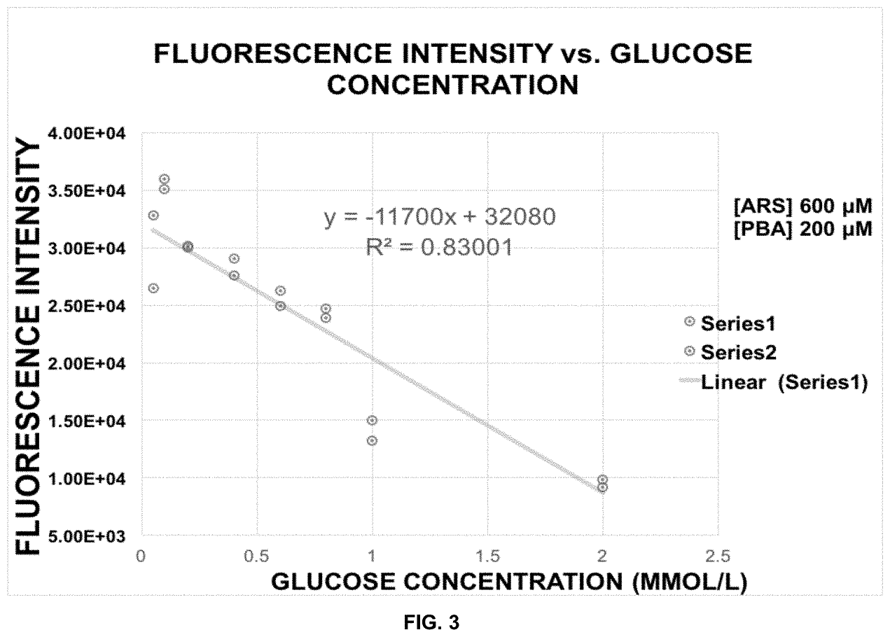

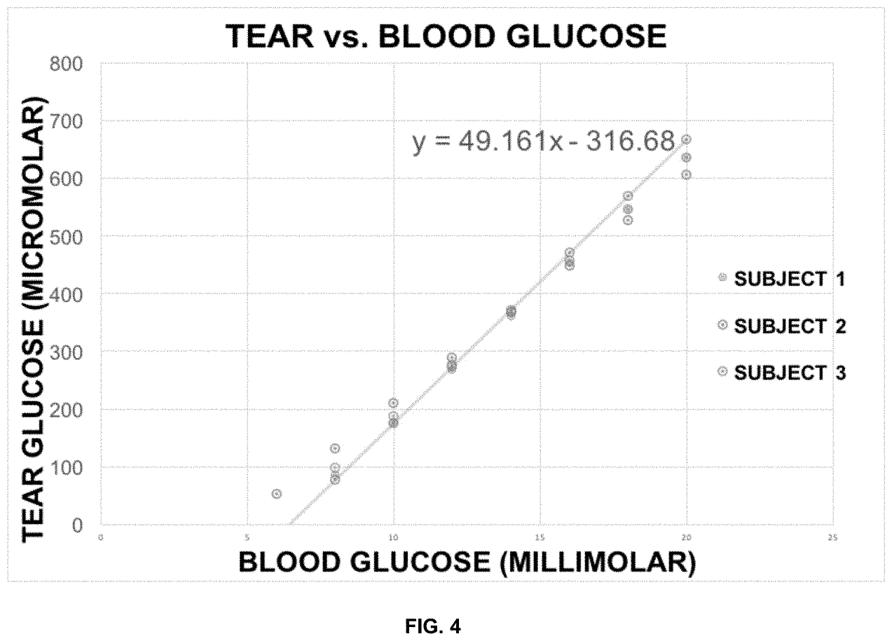

Because glucose and Alizarin Red S compete for the same diol groups on the phenylboronic acid, a decrease in the fluorescence intensity as the concentration of glucose increases occurs as shown in FIG. 3. The graph is consistent with this expectation. The fluorescence decreased from about 35K to 10K with 600 μM ARS and 200 μM PBA with glucose concentrations ranging from 0→2 mM. A multiwavelength ratioing assay in the ARS spectrum as a point of reference will be applied.

Microfluidic channels require smaller volumes of fluid for analysis and provide greater sensitivity and accuracy. In one embodiment, a plurality of hydrophobic barriers in the paper-based microfluidic device comprises a plurality of wax barriers form at least one channel. The wax barriers are produced from a hot plate or from a thermal wax, a solid ink, or a wax-based printer, a non-limiting example.

In another embodiment, a more complex, paper-based system incorporates at least one micropump to suck the fluid from the lower eyelid pouch, at least one microjet to control and steer the fluid flow and at least one microvalve to trap the fluid for analysis. In one embodiment, a 3-D printer is used to fabricate components on the paper-based microfluidic devices. In another embodiment the components, that is, the at least one microjet, the at least one valve, and the at least one pump are fabricated by cutting into the paper device using a laser cutting machine.

FIGS. 1A-1F illustrates an example embodiment of the kit. The kit comprises a handheld personal computer 20 equipped with a light source 22, a camera lens 24 and modified by a pair of selectively attachable filters, a first filter 26 configured with the light source to provide light at the excitation energy of a fluorophore complex and a second filter 28 configured to transmit light at the emission wavelength of the fluorophore complex to the camera lens. The handheld personal computer 20 can be a smartphone, a tablet computer or a personal digital assistance or other similar mobile computing devices with the features of a camera lens and a light source.

The pair of filters filter light at preset wavelengths, the first filter set at the excitation wavelength and the second filter set at the emission wavelength for a fluorophore complex of interest. These filters 26, 28 when placed over the camera lens 24 and LED light source 22 of the handheld personal computer 20 that has a software app for interpreting emission images, create a filter fluorometer.

As is understood by a person having ordinary skill in the art, in fluorometry, the light from an excitation source, such as a LED light source 22 in a handheld personal computer 20 in this instance, passes through a first filter 26 and engages the sample. The filtered light at a specific wavelength is absorbed by the sample, and molecules in the sample fluoresce. The fluorescent light transmits through a second filter 28, and light at a specific emission wavelength transmits to a means of detection, which in this example embodiment is the camera lens 24 in the modified handheld personal computer 20.

In the present disclosure, the camera lens 24 in the handheld personal computer 20 captures the image created by the emitted light. The image is further analyzed by the software app that calculates the amount of glucose in the sample based on a calibration curve.

The kit further contains a glucose-specific binding complex shown in FIGS. 1E and 1F that fluoresces when bound to glucose and a device constructed from cellulosic material having a microfluidic channel configured for wicking tear fluid from a patient's eye, example embodiments shown in FIGS. 1A-1C.

In another embodiment of the kit, the kit comprises the pair of light filters 26, 28 set to filter light at the excitation and emission wavelengths of the fluorophore-glucose complex, fluorophore glucose binding complex and a sampling device.

In another embodiment of the kit, the sampling device is a laser-cut microfluidics paper sampling device 10, 12 14. In a further embodiment, the microfluidics paper sample device includes the fluorophore glucose binding complex.

The system has the device 10, 12 14 having a microfluidic channel configured for wicking tear fluid from a patient's eye, a glucose-specific binding complex that fluoresces when bound to glucose and a fluorescence measuring device.

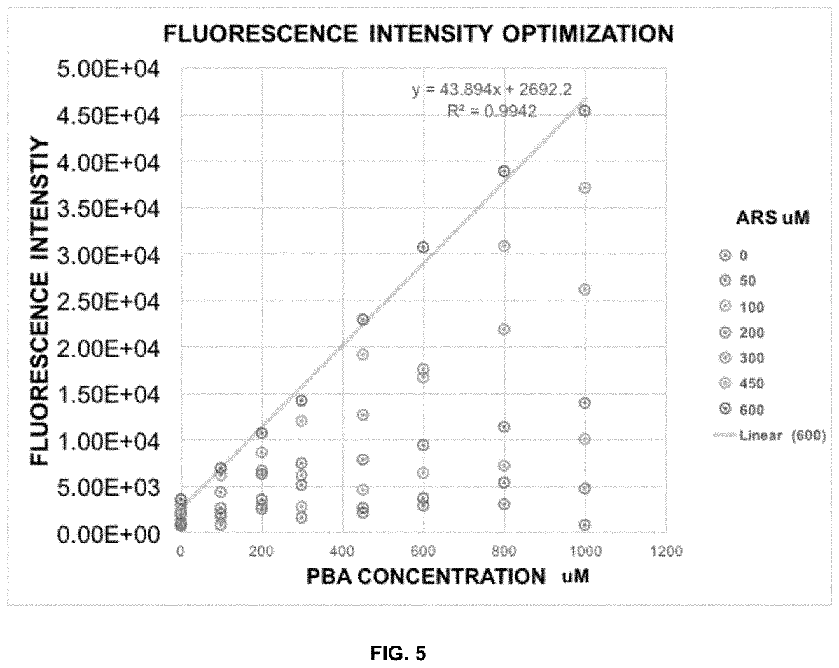

In one example embodiment, the glucose-specific binding complex is preferably a combination of Alizarin Red S and phenylboronic acid dissolved in phosphate-buffered saline. The concentration of Alizarin Red S in phosphate buffered saline ranges from 50 μM and 600 μM and the concentration of phenylboronic acid in phosphate-buffered saline ranges from 100 μM and 1000 μM. FIG. 5 plots the data for the reagents, showing the optimal concentration of Alizarin Red S in phosphate buffered saline ranges is 600 μM and phenylboronic acid is 1000 μM.

In one example embodiment, the device 10, 12, 14 having at least one microfluidic channel is preferably constructed from cellulosic material. The microfluidic channel has a width ranging from 300 μm to 550 μm. The microfluidic channels in the device are arranged as a plurality of spokes 18 radiating from a center. The microfluidic channel has at least one hydrophilic barrier.

The fluorescence measuring device is preferably the handheld personal computer 20 equipped with the camera lens 24, the light source 22 and the pair of filters 26, 28 as described hereinabove. Because the excitation wavelength and the emission wavelength are known for a specific glucose-fluorophore complex, filters 26, 28 that filter light at those wavelengths are chosen to modify the handheld personal computer 20.

The handheld personal computer 20 has a software application for measuring the intensity of the fluorescence image captured by the camera in the handheld personal computer.

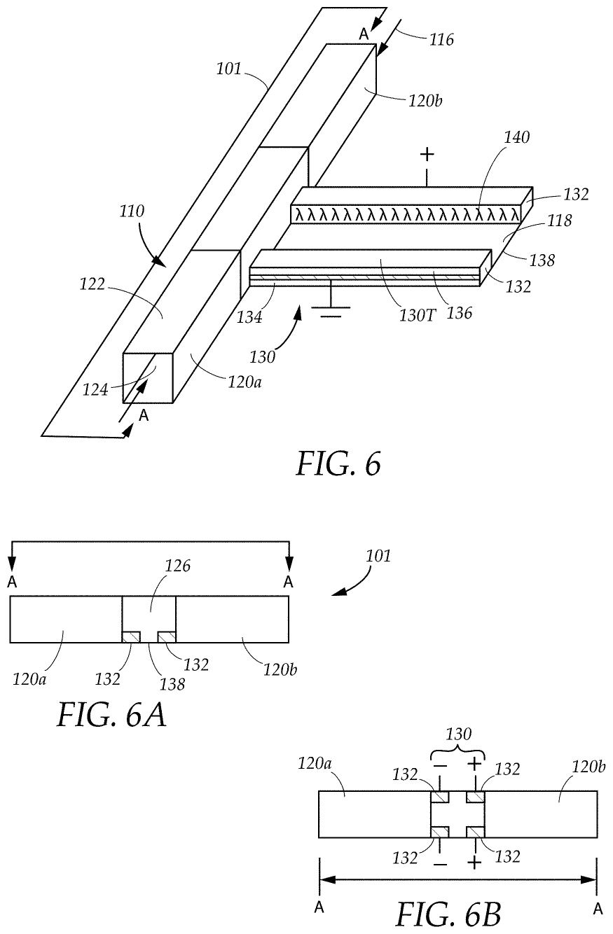

Referring to FIGS. 6-6B, a structure of the example embodiment of the microfluidic device previously discussed with regard to FIG. 1A, is shown in more detail. This example embodiment is disclosed with regard to the detection of the presence of severe acute respiratory syndrome coronavirus 2, hereafter referred to as the SARS-CoV-2 virus, but it is understood by those of ordinary skill in the art that this structure is useful for the detection of other pathogenic antigens as well as other analytes that bind with antibodies in the presence of a non-uniform electric field gradient. Nasal fluid and nasopharyngeal fluid, hereafter referred to simply as nasal fluids, are the usual fluids tested for the presence of the SARS-COV-2 virus but the method is not limited to these fluids. It understood that the term “nasal fluid” refers to both nasal fluid, nasopharyngeal fluid and similar fluids found in the human upper respiratory system.

FIG. 6 demonstrates the simplest structure of a microfluidic device with an integrated electrode, the T-channel device 100. The T-channel device is in the shape of a “T” with a stem channel 112 and a top bar 110. The top bar 110 has a pair of identical fluid channels 120a and 120b in fluid communication, the identical fluid channels 120a and 120b having an exterior channel surface 122 and an interior channel surface 124. These fluid channels 120a and 120b introduce the sample fluid in at least one channel 120a, 120b and the fluid channels 120a 120b are fluidly connected to and are in fluid communication with the stem channel 112. The at least one channel 120a, 120b is available to introduce other fluids such as isotonic saline or reagents into the device. The flow of the fluids is shown by arrows 116. It is understood by those of ordinary skill that the drawings are not proportionally drawn and that different sections are presented on a different scale in order to show detail without losing the relationship of the various components.

The exterior channel surface 122 of the at least one channel is preferably constructed from a combination of PDMS (polydimethylsiloxane also known as dimethylpolysiloxane or dimethicone) and paper materials. Unless otherwise noted, this combination is used for all fluid channels exterior channel surfaces 122 shown in the other figures unless otherwise noted. However, carbon nanotube, fibers, or polymers are possible within the inventive concept. Each channel has the interior channel surface 124.

The at least one channel 120a, 120b is soft, flexible and narrow and can be introduced directly into the nose or throat for sampling of fluid. This is because the materials of construction are soft and flexible. Advantageously, because the at least one channel is soft, flexible and narrow, sampling is more comfortable to a patient.

On the stem channel 112 of the T-channel microfluidic device 101 is an electrode pair 130 having two members 132 inside a stem channel 126, similarly constructed as the at least one fluid channel 120a, 120b described hereinabove. Only the interior of the stem channel 126 is shown in FIG. 6 in order to more clearly show the details of the electrode pair 130. The electrode pair 130 is situated on the two opposing sides of the electrode pair channel 126 with a neutral space 138 therebetween. A plurality of antibodies 140 are trapped on an interior surface of the electrode pair 130.

As fluid is pumped into the electrode pair channel 126 and a voltage applied as explained in detail herebelow, the plurality of antibodies 140 are trapped on the electrode pair 130 due to the application of a spatially non-uniform electric field gradient that is applied on the electrode pair 130 surface in a process known as positive dielectrophoresis (DEP). DEP does not require a particle to be charged. All polarizable particles exhibit dielectrophoretic activity in the presence of electric fields. A portion of the plurality of antibodies 140 also bind to the chemically treated paper insert 118. The plurality of antibodies 140 and antigens are then pulled to the electrode surface by the higher DEP force. Viral antigens bind to the antibodies 140 separating from the fluid thereby signally that the virus is present as evidenced by the detection of the antigens on the electrode pair 130.

Each electrode pair member 132 of the electrode pair 130 has an insulator layer 136, preferably composed of nitride layer below the surface 130T and a channel base 134 below an electrode pair member 132. In the drawing, the electrode pair member 132 of the electrode pair 130 towards the bottom of the FIG. 1s labeled ground and the member of the electrode pair 130 towards the top of the FIG. 1s labeled with a positive charge. It is understood by those of ordinary skill in the art that this is an arbitrary designation and that these designations are reversible. It is further understood by those of ordinary skill in the art that the electrode pair 130 is preferably in stem channel 126 but that one or more of the electrode pair 130 is possible in the other fluid channels 120 within the device and that one or more of the electrode pair 130 is possible within the same channel as explained hereinbelow.

FIG. 6A is a cross-section view of the T-channel device 101 showing the electrode pair members 132 of the electrode pair within the electrode pair channel 126 connecting to and in fluid communication with the two fluid channels 120a and 120b. FIG. 6B shows the same view of another example embodiment having a second electrode pair 130 within the same electrode pair channel. As explained hereinabove, it is understood by those of ordinary skill in the art that the designation of positive and ground is an arbitrary designation and that these designations are reversible. However, the ground of the electrode pair members 132 of the electrode pairs 130 are adjacent to each other.

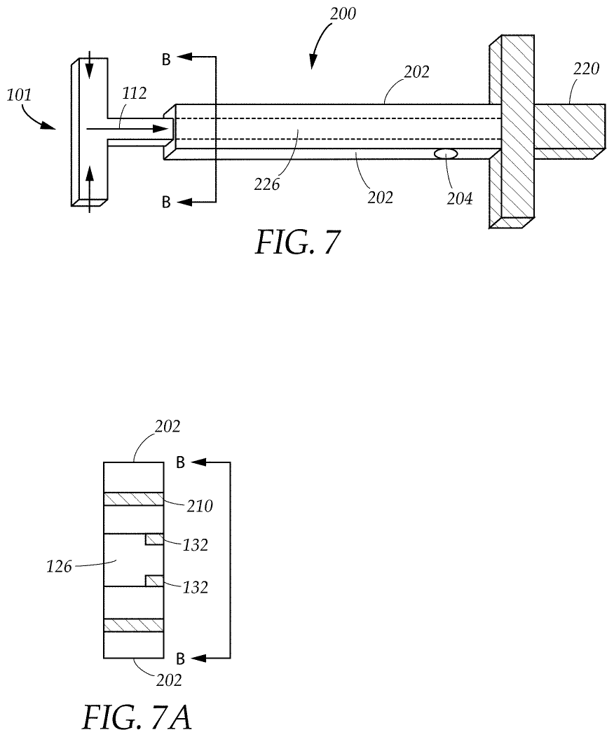

In FIGS. 7-7A, the T-shaped microfluidic device 101 selectively couples with a longer channel device 200 at a first end and having an aspirator 220 at a second end opposite the T-shaped microfluidic device and T-shaped microfluidic device 101 is fluidly connected and is in fluid communication with the longer channel device 200. The flow of fluids is by electrokinetic techniques such as dielectrophoresis, electroosmosis, and electrophoresis. The aspirator 220 manually assists the flow of fluids through the T-shaped microfluidic device 101 and the longer channel device 200 by mechanical pumping. The longer channel device 200 has a plurality of walls 202 preferably constructed from PDMS. The longer channel device is constructed to be soft, flexible, and narrow for insertion into a nose.

A cross-section shown in FIG. 7A, shows the electrode pair channel 126 inside the walls 202 of the longer channel. The T-shaped microfluidic device 101 selectively couples to the longer channel device 200 by at least one layer of non-permanent adhesive 210. The longer channel device 200 has at least one long channel 226 that runs its length, and well as a channel with a port 204 in the channel wall through the channel wall perpendicular the one long channel to remove and analyze fluid samples.

The T-shaped microfluidic device 101 when coupled to the longer channel device 200 operates by pumping fluid through the various channels 120a, 120b, 126 via a combination of mechanical pumping provided by the aspirator 220 and electroosmosis. Fluids are sucked into the various channels 120a, 120b, 126 coated with immobilized antibodies specific to SARS CoV-2. Target antigens including the SARS-CoV-2 virus bind to the immobilized antibodies 140 as described hereinabove.

The T-shaped microfluidic device 101 when coupled to the longer channel device 200 has a separate side channel with a port 204 for fluorescent detection. This separate channel contains an immobilized antibody with a fluorophore complex specific to the SARS-CoV-2 or other virus, and upon mixing with nasal fluid the fluorescent signal is detectable when SARS-CoV-2 is present.

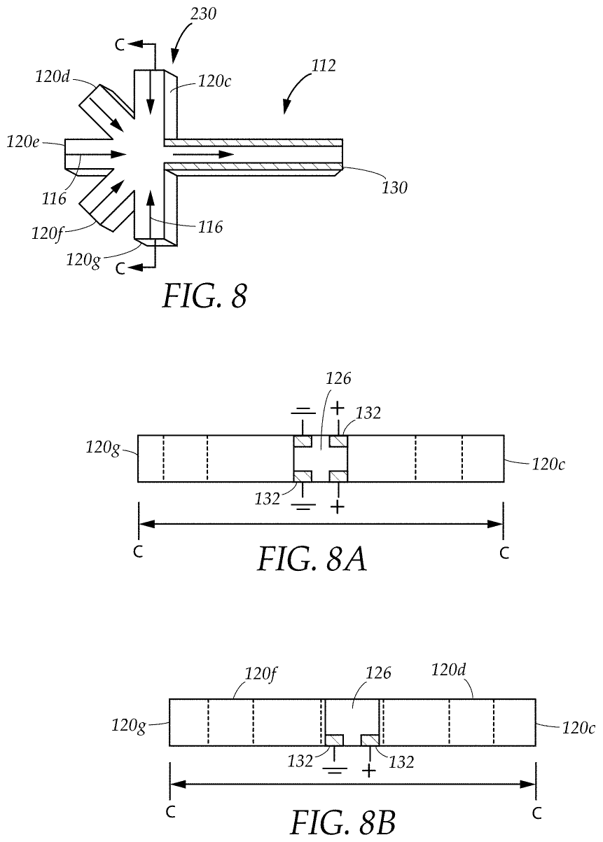

Referring to FIGS. 8-8B, a structure of the example embodiment of the microfluidic device previously discussed with regard to FIG. 1B, is shown in more detail. As disclosed hereinabove with regard to FIGS. 6-6B, the example embodiment is disclosed with regard to the detection of the presence of SARS-COV-2 virus.

FIG. 8 demonstrates a fan-shaped structure of a microfluidic device with an integrated electrode, the fan-shaped channel device 230. The fan-shaped channel device 230 is in the shape of a fan with a stem channel 112 and a plurality of identical fluid channels 120c, 120d, 120e, 120f, 120g set apart at around 45° (forty-five degrees) in a fan pattern. Fluid channels 120c and 120g form a straight line and are at a right angle with the stem channel 112. This plurality of identical fluid channels 120c, 120d, 120e, 120f, 120g is available to introduce the sample fluid in at least one channel and to introduce other fluids such as isotonic saline or reagents in at least one channel into the device. The fluid channels 120c, 120d, 120e, 120f, 120g and the electrode pair channel are in fluid communication with each other. The flow of the fluids is shown by arrows 116.

The construction of the plurality of identical fluid channels 120c, 120d, 120e, 120f, 120g is as described above with regard to the fluid channels 120a, 120b all having the same interior and exterior and are soft, flexible and narrow and can be introduced directly into the nose or throat for sampling of fluid.

On the stem channel 112 of the fan-shaped channel microfluidic device 230 is the electrode pair 130 as described hereinabove with regard to FIG. 6.

FIG. 8A-8B are cross-section views of the fan-shaped channel device 230. FIG. 8A shows a first electrode pair members 132 and a second pair of electrode pair members 132 within the same electrode pair channel 126 and functions as described hereinabove with regard to FIGS. 6-6B. FIG. 8B shows a single electrode pair within the same electrode pair channel 126. The fluid channels 120c, 120d, 120f and 120g are fluidly connected and in fluid communication with the electrode pair channel 126. It is understood by those of ordinary skill in the art that the fluid channel 120e connects directly with the electrode pair channel and is not visible in this view.

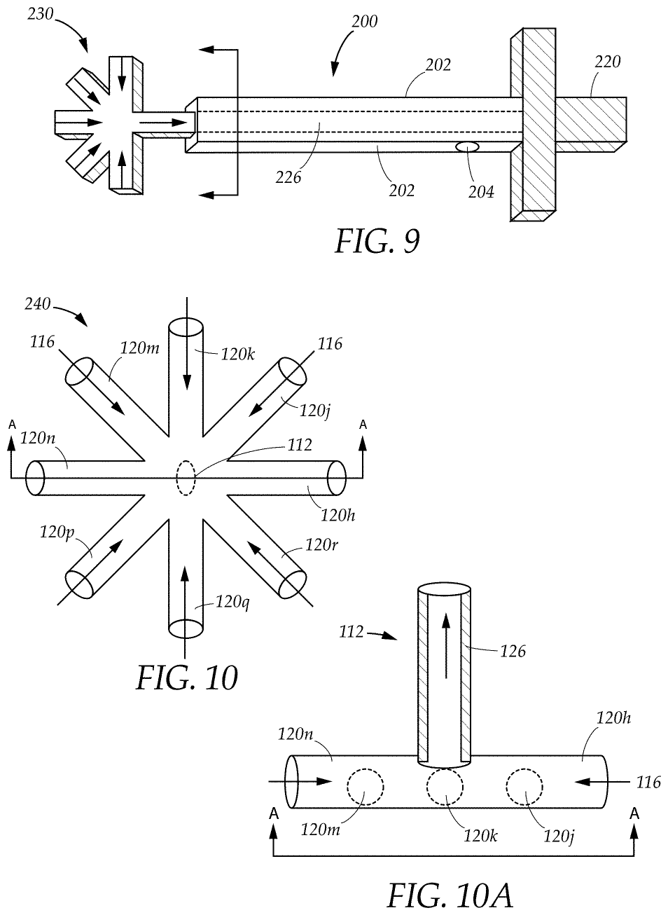

In FIG. 9, the fan-shaped microfluidic device 230 selectively couples with a longer channel device 200 having an aspirator 220 opposite the T-shaped microfluidic device. As described hereinabove with regard to FIG. 7-7A, the fan-shaped microfluidic device 230 is in fluid communication with the longer channel device 200. The aspirator 220 manually assists the flow of fluids through the fan-shaped microfluidic device 230 and the longer channel device 200 by mechanical pumping.

The fan-shaped microfluidic device 230 when coupled to the longer channel device 200 operates by pumping fluid through the various channels 120c, 120d, 120e, 120f, 120g, and 126 via a combination of mechanical pumping provided by the aspirator 220 and electroosmosis. Fluids are sucked into the various channels 120c, 120d, 120e, 120f, 120g, and 126 coated with immobilized antibodies specific to SARS CoV-2. Target antigens including the SARS-CoV-2 virus bound to the immobilized antibodies 140 as described hereinabove.

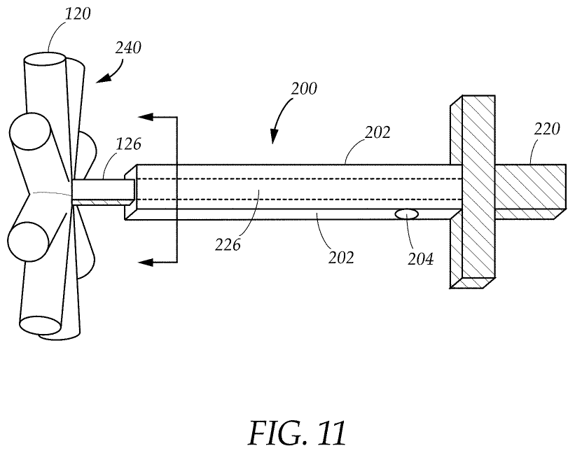

Referring to FIGS. 10-10A, a structure of the example embodiment of the microfluidic device previously discussed with regard to FIG. 10, is shown in more detail. As disclosed hereinabove with regard to FIGS. 6-6B, the example embodiment is disclosed with regard to the detection of the presence of SARS-COV-2 virus.

FIGS. 10-10A demonstrates a spoke-shaped structure of a microfluidic device with an integrated electrode, the spoke-shaped channel device 240. The spoke-shaped channel device 240 is in the shape of the spokes of a wagon wheel with a stem channel 112 in a plane orthogonal to the plane of the spokes. A plurality of identical fluid channels 120h, 120j, 120k, 120m, 120n, 120p, 120q and 120r are in the same plane and are set apart at around 45° (forty-five degrees) in the spoke pattern. The stem channel 112 is in a plane at right angle to the plane of the plurality of identical fluid channels 120h, 120j, 120k, 120m, 120n, 120p, 120q and 120r. This plurality of identical fluid channels 120h, 120j, 120k, 120m, 120n, 120p, 120q and 120r is available to introduce the sample fluid in at least one channel and to introduce other fluids such as isotonic saline or reagents in at least one channel into the device. The fluid channels 120h, 120j, 120k, 120m, 120n, 120p, 120q and 120r and the electrode pair channel 126 in the stem channel 112 are in fluid communication with each other. The flow of the fluids is shown by arrows 116.

The construction of the plurality of identical fluid channels 120h, 120j, 120k, 120m, 120n, 120p, 120q and 120r is as described above with regard to the fluid channels 120a, 120b all having the same interior and exterior and are soft, flexible and narrow and can be introduced directly into the nose or throat for sampling of fluid.

On the stem channel 112 of the spoke-shaped channel microfluidic device 240 is the electrode pair 130 as described hereinabove with regard to FIG. 6.

FIG. 10A is a cross-section view of the spoke-shaped channel device 240 along the x, y, z axes.

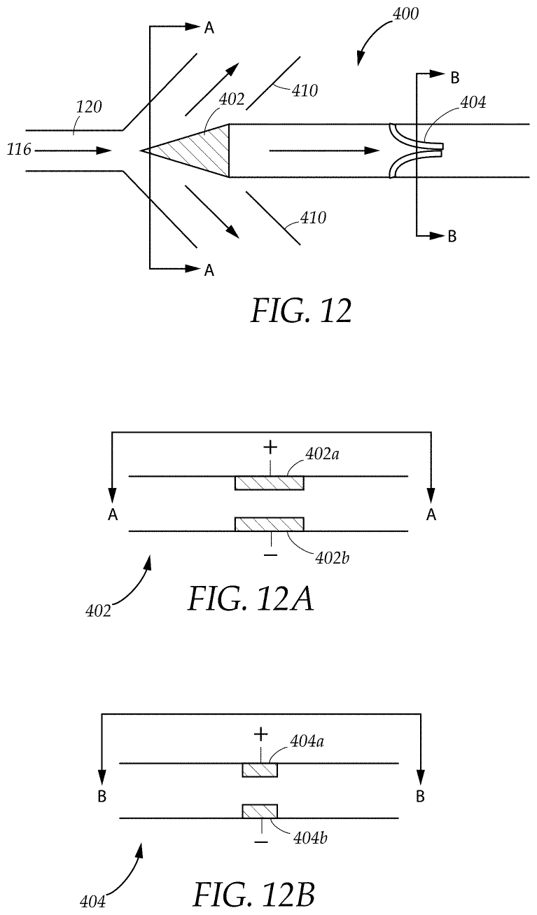

In FIG. 11, the spoke-shaped microfluidic device 240 selectively couples with a longer channel device 200 having an aspirator 220 opposite the T-shaped microfluidic device. As described hereinabove with regard to FIG. 7-7A, the spoke-shaped microfluidic device 240 is in fluid communication with the longer channel device 200. The aspirator 220 manually assists the flow of fluids through the fan-shaped microfluidic device 240 and the longer channel device 200 by mechanical pumping.

The spoke-shaped microfluidic device 240 when coupled to the longer channel device 200 operates by pumping fluid through the various channels 120, via a combination of mechanical pumping provided by the aspirator 220 and electroosmosis. Fluids are sucked into the various channels 120 and 126 coated with immobilized antibodies specific to SARS CoV-2. Target antigens including the SARS-CoV-2 virus bound to the immobilized antibodies 140 as described hereinabove.

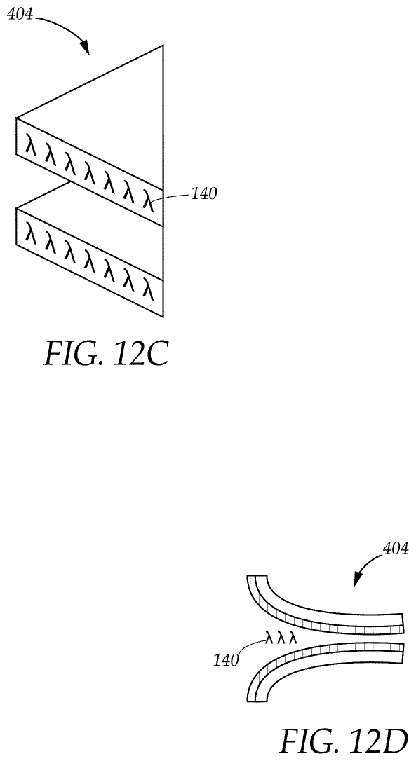

FIGS. 12-12D demonstrate a novel filtering electrode system 400 within a microfluidic system that also provides filtration in addition to detection. While these FIGURES show a single fluid channel 120 with the direction of the fluid flow shown by arrows 116, it is understood by those of ordinary skill in the art that this electrode system can be substituted for the electrode pair described hereinabove in all three microfluidic systems. In the drawings, a triangular electrode 402 and a curved electrode pair 404 are shown, but it is further understood that other geometric shapes for these electrodes pairs are envisioned within the inventive concept.

The sample fluid with a plurality of biological particles including the analytes, in particular the analytes that are antigens as described hereinabove enters the fluid channel 120 and flows towards the triangular electrode 402 having an electrode pair 402a, 402b. When the voltage is applied at a specified frequency, the antibodies 140 in combination with the antigen combination are trapped on the energized electrode due to positive dielectophoresis and the remaining fluid and biological particles filter out and exit through a plurality of side channels 410. When the voltage on the triangular electrode 402 is turned off, the antibodies 140 in combination with the antigens are then able to move towards a curved electrode pair 404. Voltage is applied to the curved electrode pair 404. The antibodies 140 in combination with the antigens are trapped on the energized curved electrode pair 404. Upon turning off the voltage, the antibody-antigen combination is released for further determinations.

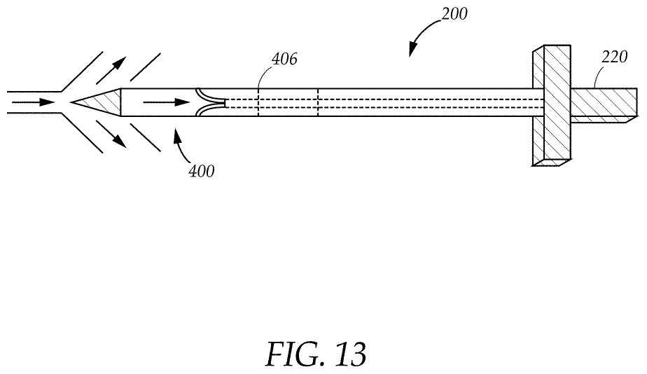

In FIG. 13, the filtering electrode system is selectively coupled to the longer channel device 200 and aspirator 220 at a junction 406. The filtering electrode system is in fluid communication with the longer channel device 200 as explained hereinabove.

It is understood that when an element is referred hereinabove as being “on” another element, it can be directly on the other element or intervening elements may be present therebetween. In contrast, when an element is referred to as being “directly on” another element, there are no intervening elements present.

Moreover, any components or materials can be formed from a same, structurally continuous piece or separately fabricated and connected.

It is further understood that, although ordinal terms, such as, “first,” “second,” “third,” are used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

Spatially relative terms, such as “beneath,” “below,” “lower,” “above,” “upper” and the like, are used herein for ease of description to describe one element or feature's relationship to another element(s) or feature(s) as illustrated in the figures. It is understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

Example embodiments are described herein with reference to cross section illustrations that are schematic illustrations of idealized embodiments. As such, variations from the shapes of the illustrations as a result, for example, of manufacturing techniques and/or tolerances, are to be expected. Thus, example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

In conclusion, herein is presented a noninvasive method for measuring blood glucose levels. The disclosure is illustrated by example in the drawing figures, and throughout the written description. It should be understood that numerous variations are possible, while adhering to the inventive concept. Such variations are contemplated as being a part of the present disclosure.

What is claimed is:

1. A microfluidic device for detecting a presence of SARS-CoV-2 virus, comprising:
a plurality of microfluidic fluid channels in fluid communication with an electrode pair channel;
an electrode channel having at least one electrode pair within, the at least electrode pair having a first electrode pair member and a second electrode pair member; and
a plurality of antibodies configured to bind with a plurality of SARS-CoV-2 virus antigens by positive dielectrophoresis in a presence of an electric field created by the at least one electrode pair.

2. The microfluidic device as described in claim 1, wherein the plurality of antibodies are trapped on the electrode pair when a spatially non-uniform electric field gradient is applied through positive dielectrophoresis.

3. The microfluidic device as described in claim 2, further comprising a chemically treated paper insert between a first member of the at least one electrode pair and a second member of the at least one electrode pair.

4. The microfluidic device as described in claim 3, wherein a portion of the plurality of antibodies bind to the chemically treated paper insert.

5. The microfluidic device as described in claim 4, wherein the plurality of microfluidic fluid channels are soft, flexible and narrow.

6. The microfluidic device as described in claim 5, wherein the plurality of microfluidic fluid channels are constructed from polydimethylsiloxane and paper.

7. The microfluidic device as described in claim 6, wherein the plurality of microfluidic fluid channels form a T-shaped microfluidic device when fluidly connected to the electrode channel.

8. The microfluidic device as described in claim 6, wherein the plurality of microfluidic fluid channels form a fan-shaped microfluidic device when fluidly connected to the electrode channel.

9. The microfluidic device as described in claim 6, wherein the plurality of microfluidic fluid channels form a spoke-shaped microfluidic device in a first plane the microfluidic channels fluidly connected to the electrode channel, the electrode channel in a second plane orthogonal to the first plane.

10. A system for detecting a presence of SARS-CoV-2 virus in fluid, comprising:
a microfluidic device having an electrode channel having at least one electrode pair within, the electrode pair having a first electrode pair member and a second electrode pair member, a plurality of microfluidic fluid channels in fluid communication with the electrode pair channel and a plurality of antibodies configured to bind with a plurality of SARS-CoV-2 virus antigens by positive dielectrophoresis in the presence of an electric field created by the at least one electrode pair;
a longer channel device in fluid communication with the microfluidic device, having a pair of ends, the first end selectively connecting to the microfluidic device; and
an aspirator connecting to a second end of the longer channel device.

11. The system as described in claim 10, wherein the plurality of antibodies in the microfluidic device are trapped on the at least one electrode pair when a spatially non-uniform electric field gradient is applied through the positive dielectrophoresis.

12. The system as described in claim 11, wherein the longer channel device further comprises a channel wall and at least one long channel inside the channel wall extending through the length of the longer channel device, the at least one long channel fluidly connecting to the electrode channel of the microfluidic device, the longer channel device adhesively connecting to the microfluidic device.

13. The system as described in claim 12, wherein the longer channel device further comprises a port in the channel wall perpendicular to the at least one channel for removing fluid for further analysis.

14. The system as described in claim 13, wherein fluid moves through the microfluidic device into the longer channel device through by electroosmosis and manually pumping.

15. The system as described in claim 14, wherein the aspirator provides manual pumping.

16. The system as described in claim 15, wherein the plurality of antibodies in the microfluidic device are trapped on the electrode pair when the spatially non-uniform electric field gradient is applied through positive dielectrophoresis.

17. The system as described in claim 16, wherein the longer channel device and the plurality of fluid channels of the microfluidic device are constructed from polydimethylsiloxane and paper.

18. The system as described in claim 17, wherein the longer channel device and the plurality of microfluidic fluid channels of the microfluidic device are soft, flexible and narrow.

19. The system as described in claim 18, further comprising a chemically treated paper insert between a first member of the at least one electrode pair and a second member of the at least one electrode pair, wherein a portion of the plurality of antibodies bind to the chemically treated paper insert.

20. A filtering microfluidic device for detecting a presence of SARS-CoV-2 virus, comprising:
an electrode pair channel having at least one pair of triangular electrodes and one pair of curved electrodes;
at least one fluid channel in fluid communication with the electrode pair channel; and
a plurality of antibodies configured to bind with a plurality of SARS-CoV-2 virus antigens by positive dielectrophoresis in a presence of an electric field such that when a voltage is applied to the filtering microfluidic device the plurality of antibodies in combination with the plurality of SARS-CoV-2 virus antigens are trapped on the triangular electrode pair due to positive dielectophoresis, allowing the remaining fluid to exit and when the voltage on the triangular electrode is turned off the plurality of antibodies in combination with the plurality of SARS-CoV-2 virus antigens then move towards the curved electrode pair and when the voltage is applied to the curved electrode pair the plurality of antibodies in combination with the plurality of SARS-CoV-2 virus antigens trapped on the energized curved electrode pair.
